(19) United States
(12) Patent Application Publication
(10) Pub. No.: US 2023/0240613 A1
(43) Pub. Date: Aug. 3, 2023

(54) Pedal Pulse Monitoring Device

(71) Applicants: Jordan Aguilar, Omak, WA (US); Alexander Aguilar, Omak, WA (US)

(72) Inventors: Jordan Aguilar, Omak, WA (US); Alexander Aguilar, Omak, WA (US)

(21) Appl. No.: 18/155,377

(22) Filed: Jan. 17, 2023

Related U.S. Application Data
(60) Provisional application No. 63/304,738, filed on Jan. 31, 2022.

(51) Int. Cl. A61B 5/00; A61B 5/0205

(52) U.S. Cl. CPC A61B 5/6829; A61B 5/02055; A61B 5/743; A61B 5/746; A61B 2562/0271

(57) ABSTRACT

A pedal pulse monitoring device for use in monitoring a pulse of a patient. The device is comprised of a display and at least one pad in electrical communication with the display via at least one wire or wireless technology. The pad can be secured to a toe(s) and/or a foot(s) of the patient to read the pedal pulse rate, pedal pulse quality, and pedal temperature of the patient. The pad then communicates the pedal pulse rate, pedal pulse quality, and pedal temperature to the display. The pedal pulse quality is displayed on the display as absent, weak, or bounding and/or with a pulse wave analysis. The display further alerts a medical provider if the patient's pedal pulse rate, pedal pulse quality, and/or pedal temperature go outside of customizable parameters set by medical professionals via a speaker and/or a light.

### CROSS-REFERENCE TO RELATED APPLICATION

The present application claims priority to, and the benefit of, U.S. Provisional Application No. 63/304,738, which was filed on Jan. 31, 2022, and is incorporated herein by reference in its entirety.

### FIELD OF THE INVENTION

The present invention relates generally to the field of pulse monitoring devices. More specifically, the present invention relates to a pedal pulse monitoring device primarily comprised of a display and at least one pad in electrical communication with the display via at least one wire or wireless technology. The pad(s) can be secured to the toe and/or the foot of the patient to read the pedal pulse rate, pedal pulse quality and pedal temperature of the patient. The pad(s) then communicates the pedal pulse rate, pedal pulse quality and pedal temperature to the display monitor. The display further alerts a medical provider if the patient's pedal pulse rate increases, decreases, if the pedal pulse quality becomes absent, weak, or bounding, and/or if the pedal temperature changes outside of customizable parameters set by a medical professional via a speaker and/or a light. Accordingly, the present disclosure makes specific reference thereto. Nonetheless, it is to be appreciated that aspects of the present invention are also equally applicable to other like applications, devices, and methods of manufacture.

### BACKGROUND

Finding and assessing a pedal pulse is part of a routine nursing assessment. Assessing a pedal/lower extremity pulse is a frequent part of the ongoing assessment for a patient with an injury, surgery, condition, or anything affecting blood flow to the lower extremities. However, locating the pedal pulse of a medical patient can be extremely difficult and time-consuming, especially when both legs and/or extreme injury are involved. Performing frequent and continual monitoring of a pedal pulse is nearly impossible, which delays patient care and puts increased demand on the medical staff.

Therefore, there exists a long-felt need in the art for a pedal pulse monitoring device. There also exists a long-felt need in the art for a pedal pulse monitoring device that provides medical personnel with an automated pedal pulse monitoring device that displays vital information on a highly visible display unit. Further, there exists a long-felt need in the art for a pedal pulse monitoring device that provides continual monitoring of pedal pulse rate, pedal pulse quality, and pedal temperature to aid in early recognition of patient condition or changes. In addition, there exists a long-felt need in the art for a pedal pulse monitoring device that offers continual monitoring to eliminate the need for medical personnel to perform frequent manual assessments of a patient's pedal pulse rate, pedal pulse quality, and pedal temperature. This also assists in tracking data for quality improvement measures for patient care and allows for instant alarm notification of a change in a patient's pedal pulse data.

The subject matter disclosed and claimed herein, in one embodiment thereof, comprises a pedal pulse monitoring device. The device is primarily comprised of a display and at least one pad in electrical communication with the display via at least one wire or wireless technology. The pad(s) can be secured to one or both feet and/or toes of the patient such that the pad(s) reads the pedal pulse rate, pedal pulse quality and pedal temperature of the patient. The pad(s) then communicates the pedal pulse rate, pedal pulse quality, and pedal temperature to the display. The display further alerts a medical provider via a speaker and/or a light if the patient's pedal pulse quality is absent, weak, bounding, if the pedal pulse rate is rapidly increasing or decreasing, and/or if the pedal temperature changes outside of customizable parameters set by a medical professional.

In this manner, the pedal pulse monitoring device of the present invention accomplishes all of the foregoing objectives and provides a pulse monitoring device that displays vital information on a highly visible display unit in an automatic manner. The device also provides continual monitoring of pedal pulse rate, pedal pulse quality, and pedal temperature to aid in early recognition of patient condition or changes. In doing so, the device eliminates the need for medical personnel to perform frequent manual assessments of a patient's pedal pulse data.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a pedal pulse monitoring device. The device is primarily comprised of a display and at least one pad in electrical communication with the display via at least one wire or wireless technology. The display may be any display screen type such as, but not limited to, a touch screen, an LCD screen, an OLED screen, an ELD screen, an LED-backlit LCD screen, an LED screen, a PDP screen, etc. In one embodiment, the display is enclosed by a splash resistant protective shell, preferably comprised of at least one fastener mount that allows the device to easily attach to a hospital bed or other item, surface, or location. In this manner, the display can be mounted as needed to be easily viewed by medical professionals. The display is also comprised of at least one charging port that receives at least one reciprocating power cord from at least one charger of the device or with battery powered technology. In this manner, the charging port allows the charger to supply power to the battery of the display to power the display.

The device is also comprised of at least one pad comprised of any medical-grade material known in the art. The pad(s) are applied to a foot and/or toe of a patient, preferably in the location of the foot that allows the pad to monitor the pedal pulse rate, pedal pulse quality, and/or pedal temperature of the patient such as, but not limited to, the toe and/or dorsalis pedis pulse locations. The pad is preferably comprised of a bottom surface, wherein the bottom surface is comprised of a skin-safe adhesive coating or other medical-grade adhesive coating known in the art. As a result, the pad remains secured to the foot during use.

The pad is also preferably comprised of at least one pulse oximetry sensor, preferably comprised of at least one electrode that detects the pedal pulse rate and pedal pulse quality of the patient from the patient's foot and/or toes. The pedal pulse rate and pedal pulse quality detected are communicated from the electrode(s) to the display via the wire or wireless technology in the form of an electrical signal that is then categorized by at least one processor of the display. The processor categorizes the patient's pedal pulse quality as absent, weak, and/or bounding with the option of a pulse wave analysis that can be displayed on the display unit. The processor categorizes the pedal pulse rate in beats per minute, which is displayed on the display. In one embodiment, the display is comprised of at least one speaker and at least one light such as, but not limited to, a colored or non-colored LED. If the patient's pedal pulse rate increases, decreases, if the pedal pulse quality becomes absent, weak, or bounding, and/or if the pedal temperature changes outside of customizable parameters set by a medical professional, the speaker and/or light are activated to alert the change to a medical professional.

An additional pad is also comprised of at least one temperature sensor that measures the pedal temperature of the patient through the patient's foot. The temperature detected is communicated to the display via the wire in the form of an electrical signal. The electrical signal is then converted by at least one processor of the display into a temperature that is displayed on the display. In one embodiment, the sensors communicate with the display in a wireless manner.

Accordingly, the pedal pulse monitoring device of the present invention is particularly advantageous as it provides a pedal pulse monitoring device that displays vital information on a highly visible display unit in an automatic manner. Further, the device provides continual monitoring of pedal pulse rate, pedal pulse quality, and pedal temperature to aid in early recognition of patient condition or changes. Therefore, the device eliminates the need for medical personnel to perform frequent manual assessments of a patient's pedal pulse data. In this manner, the pedal pulse monitoring device overcomes the limitations of existing pulse monitoring devices known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a pedal pulse monitoring device of the present invention while attached to the patient's feet and a hospital bed in accordance with the disclosed architecture;

FIG. 2 illustrates a perspective view of a display, a charger, and a plurality of pads of one potential embodiment of a pedal pulse monitoring device of the present invention while attached to the patient's feet and a hospital bed in accordance with the disclosed architecture; and

FIG. 3 illustrates an enhanced perspective view of a display of one potential embodiment of a pedal pulse monitoring device of the present invention while attached to a hospital bed in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a pedal pulse monitoring device. There also exists a long-felt need in the art for a pedal pulse monitoring device that provides medical personnel with an automated pedal pulse monitoring device used that displays vital information on a highly visible display unit. Further, there exists a long-felt need in the art for a pedal pulse monitoring device that provides continual monitoring of pedal pulse rate, pedal pulse quality, and pedal temperature to aid in early recognition of patient condition or changes. There also exists a long-felt need in the art for a pedal pulse monitoring device that compares pedal pulse rate, pedal pulse quality, and/or pedal temperature between a patient's left and right feet. Also, allowing for customizable parameters to be set to alarm if a patient's pedal pulse rate pedal pulse quality, and/or pedal temperature are different in one of their feet compared to the other foot. In addition, there exists a long-felt need in the art for a pedal pulse monitoring device that offers continual monitoring to eliminate the need for medical personnel to perform frequent manual assessments of a patient's pedal pulse data. Likewise, there exists a long-felt need in the art for a pedal pulse monitoring device that can send data received from a patient's pedal sensors to a centralized hub for healthcare personnel to review and/or be alerted of the patient's pedal condition changes, as well as the patient's pedal data being saved for the electronic health record. The frequency of the monitoring intervals of the patient's pedal pulse data can also be customized

The present invention, in one exemplary embodiment, is comprised of a pedal pulse monitoring device primarily comprised of a display and at least one pad in electrical communication with the display via at least one wire or wireless technology. The display may be any display screen type such as, but not limited to, a touch screen, an LCD screen, an OLED screen, an ELD screen, an LED-backlit LCD screen, an LED screen, a PDP screen, etc. The display may be enclosed by a splash resistant protective shell preferably comprised of at least one fastener that allows the device to easily attach to a hospital bed or other item, surface, or location. In this manner, the display can be mounted as needed to be easily viewed by medical professionals. The display is also comprised of at least one charging port that receives at least one reciprocating power cord from at least one charger of the device or powered through batteries. In this manner, the charging port allows the charger to supply power to the battery of the display to power the display.

The device is also comprised of at least one pad. The pad(s) is preferably comprised of any medical grade material known in the art and can be applied to a foot(s) and/or toes of a patient. The pad(s) is preferably applied to the toes and/or dorsalis pedis pulse locations to allow the pad(s) to monitor the pedal pulse rate of the patient in beats per minute, and categorizing the pedal pulse quality through absent, weak, or bounding and/or with the option of a pulse wave analysis. The pad is preferably comprised of an adhesive bottom surface such that the pad remains secured to the foot during use.

The pad is also preferably comprised of at least one pulse oximetry sensor preferably comprised of at least one electrode that detects the pedal pulse rate and the pedal pulse quality of the patient from the patient's toes or foot. The pedal pulse rate and pedal pulse quality detected is communicated from the electrode to the display via the wire in the form of an electrical signal or wireless technology that is then categorized by at least one processor of the display. The processor categorizes the pedal pulse quality of the patient as absent, weak, and/or bounding with the option of a pulse wave analysis which is displayed on the display monitor. In one embodiment, the display is comprised of at least one speaker and at least one light such as, but not limited to, a colored or non-colored LED. If the pedal pulse rate, pedal pulse quality, and/or pedal temperature go outside of parameters preset by medical professionals, the speaker, alarm and/or light can be activated to alert a nearby medical professional.

An additional pad is also comprised of at least one temperature sensor that measures the pedal temperature of the patient through the patient's foot. The pedal temperature detected is communicated to the display via the wire in the form of an electrical signal. The electrical signal is then converted by at least one processor of the display into a temperature that is displayed either in Fahrenheit or Celsius on the display monitor. In one embodiment, the sensors communicate with the display in a wireless manner.

Accordingly, the pedal pulse monitoring device of the present invention is particularly advantageous as it provides a pulse monitoring device that displays vital information on a highly visible display unit in an automatic manner. Further, the device provides continual monitoring of pedal pulse rate, pedal pulse quality, and pedal temperature to aid in early recognition of patient condition or changes. Therefore, the device eliminates the need for medical personnel to perform frequent manual assessments of a patient's pedal pulse.

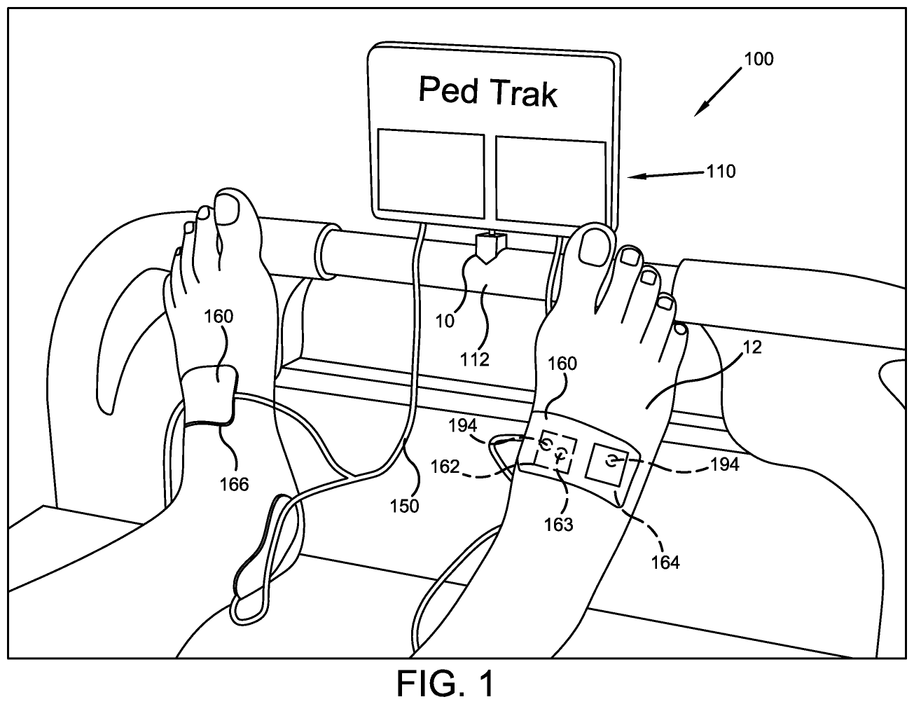

Referring initially to the drawings, FIG. 1 illustrates a perspective view of one potential embodiment of a pedal pulse monitoring device 10 of the present invention while attached to the patient's feet 12 and a hospital bed 10 in accordance with the disclosed architecture. The device 100 is primarily comprised of a display screen 110 and at least one pad 160 in electrical communication with the display 110 via at least one wire or wireless technology 150. In the preferred embodiment, the display 110 is square or rectangular in shape. However, the display 110 may be any shape known in the art such as, but not limited to, circular, polygonal, etc. The display 110 may be any display screen type such as, but not limited to, a touch screen, an LCD screen, an OLED screen, an ELD screen, an LED-backlit LCD screen, an LED screen, a PDP screen, etc. In one embodiment, the display 110 is enclosed by a splash resistant protective shell 116. The shell 116 is preferably made from a rigid plastic material such as, but not limited to, acrylic, polycarbonate, polyethylene, thermoplastic, acrylonitrile butadiene styrene, low-density polyethylene, medium-density polyethylene, high-density polyethylene, polyethylene terephthalate, polyvinyl chloride, polystyrene, polylactic acid, acetal, nylon, fiberglass, recycled plastic, biodegradable plastic, etc. The display 110 can be turned on/off via at least one button 180. Alternatively, all functions of the device 100 may be controlled by the display 110 in a touchscreen embodiment.

The shell 116 is preferably comprised of at least one fastener 112. The fastener 112 allows the device 100 to easily attach to a hospital bed 10 or another item, surface, or location. The fastener 112 may be any fastener known in the art such as, but not limited to, a clip, a clamp, hook and loop, a magnet, a suction cup, a threaded bolt, a hinge, a swiveling fastener, mount, adhesive surface, etc. In this manner, the display 110 can be mounted as needed to be easily viewed by medical professionals in the preferred location.

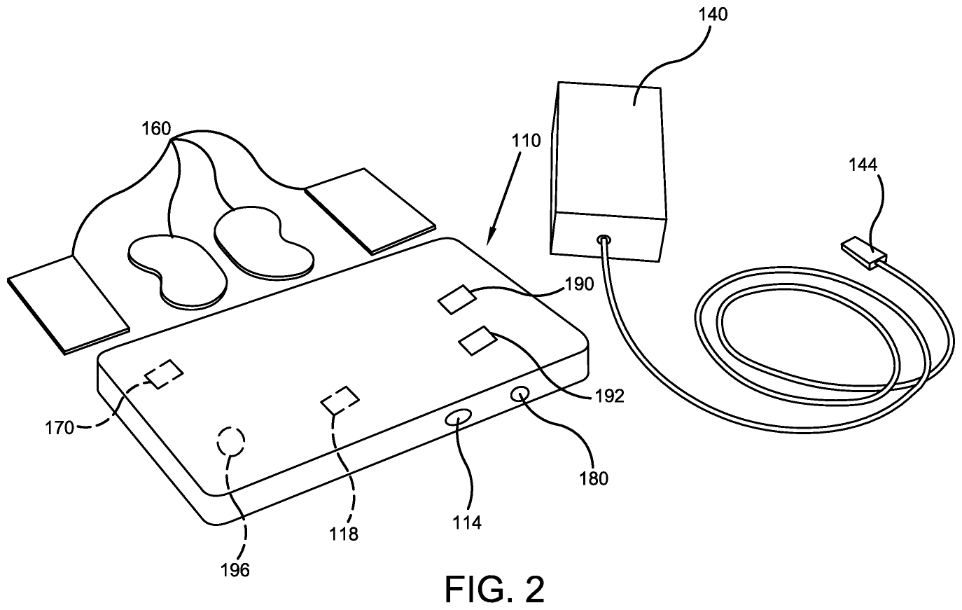

The display 110 is also comprised of at least one charging port or powered through battery power 114. The charging port 114 receives at least one reciprocating power cord 144 from at least one charger 140 of the device 100, as seen in FIG. 2. In the preferred embodiment, the port 114 and cord 144 are a reciprocating USB-A, USB-B, Micro-B, Micro-USB, Mini-USB, USB-C, or a 9 v/12 v power supply port. In this manner, the charging port 114 allows the charger 140 to supply power to the battery 118 of the display 110 to power the display 110. The charger 140 can preferably be plugged into a female 110V/240V outlet. The battery 118 may be a disposable battery 118 or a rechargeable battery 118 in the form of an alkaline, nickel-cadmium, nickel-metal hydride battery 118, etc., such as any 3V-12 volts DC battery 118 or other conventional battery 118 such as A, AA, AAA, etc., that supplies power to the device 100. Throughout this specification, the terms “battery” and “batteries” may be used interchangeably to refer to one or more wet or dry cells or batteries 118 of cells in which chemical energy is converted into electricity and used as a source of DC power. References to recharging or replacing batteries 118 may refer to recharging or replacing individual cells, individual batteries 118 of cells, or a package of multiple battery cells as is appropriate for any given battery 118 technology that may be used.

The device 100 is also comprised of at least one pad. In the preferred embodiment, the pad 160 is rectangular in shape. However, the pad 160 may be any shape known in the art such as, but not limited to, elongated ovoid circular, polygonal, etc. comprised of any medical grade material known in the art such as, but not limited to, fabric, cloth, etc. The pad(s) 160 is applied to a foot(s) and/or toe(s) 12 of a patient, preferably in the location of the foot and/or toe 12 that allows the pad 160 to monitor the pedal pulse rate and pedal pulse quality of the patient such as, but not limited to, the toe and dorsalis pedis pulse locations. The pad 160 is preferably comprised of a bottom surface 166, wherein the bottom surface is comprised of a skin-safe adhesive coating or other medical grade adhesive coating known in the art. In this manner, the pad 160 remains secured to the foot and/or toe 12 during use.

The pad 160 is also preferably comprised of at least one pedal pulse oximetry sensor 162 preferably comprised of at least one electrode 163. The pedal pulse oximetry sensor 162 detects the pedal pulse rate and pedal pulse quality of the patient from the patient's foot and/or toe 12. The pad 160 may be any shape to easily conform and attach to any area of the foot and/or toe 12. The pedal pulse rate and pedal pulse quality detected is communicated from the electrode 163 to the display 110 via the wire 150 in the form of an electrical signal or wireless technology. The wire 150 may be fixedly or removably attached to the pad 160 and the display 110. The electrical signal is then categorized by at least one processor 170 of the display 110. The processor 170 categorizes the pedal pulse quality of the patient as absent, weak, and/or bounding if any category is applicable. The processor also displays the pedal pulse rate in beats per minute. In differing embodiments, parameters can be set via the display 110 and/or button 180 for the pedal pulse rate, pedal pulse quality (absent, weak, and/or bounding), and pedal temperature. Parameters can also be set for how often the data will be recorded and sent to a centralized hub, where this data can be saved and/or viewed by medical personnel.

In one embodiment, the display 110 is comprised of at least one speaker, alarm 190 and at least one light 192 such as but not limited to a colored or non-colored LED. When the pedal pulse rate, pedal pulse quality, and/or pedal temperature moves out of parameters set by a medical professional 100, the speaker 190 and/or light 192 are activated to alert a nearby medical professional. The speaker 190 may produce at least one audible tone, sound, alarm, etc., and the light 192 may turn on or flash. The speaker 190 and/or light 192 can be activated/deactivated by the button or touch screen 180. In one embodiment, the speaker 190 and/or light 192 respond differently to each category to aid a medical professional in quickly decerning the pedal pulse condition of the patient.

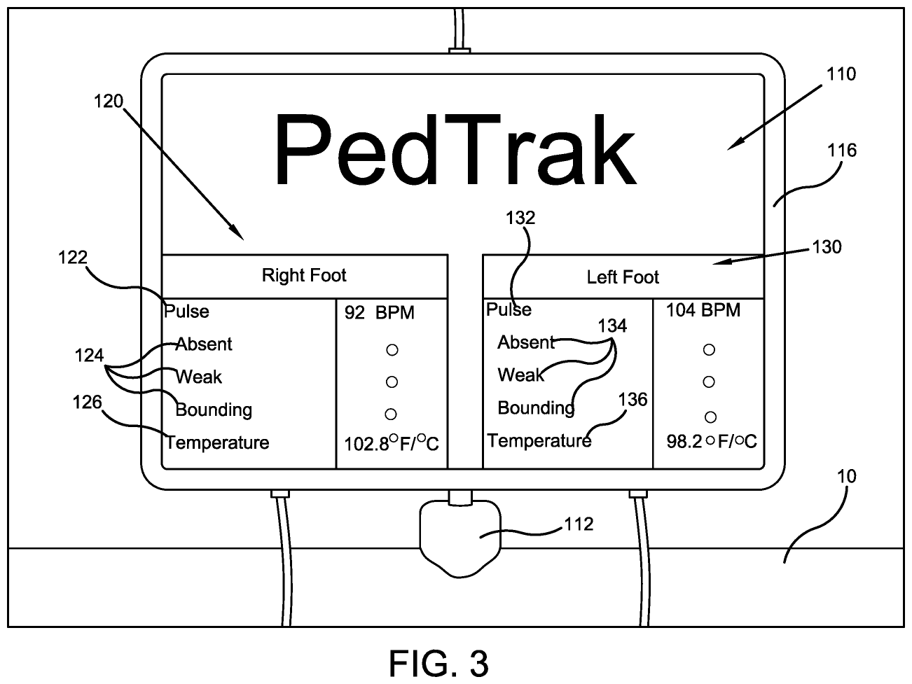

FIG. 3 illustrates an enhanced perspective view of a display 110 of one potential embodiment of a pedal pulse monitoring device 100 of the present invention while attached to a hospital bed in accordance with the disclosed architecture. The display 110 is comprised of at least a first foot display area 120 and preferably a second foot display area and can be customizable by the user 130. Both areas display the pedal pulse rate, 122,132, pedal pulse quality 124,134 (i.e., absent, weak, and/or bounding), and pedal temperature detected from each foot 12 of the patient. In this manner, the pedal pulse rate, pedal pulse quality, and pedal temperature of the patient can be easily monitored.

An additional pad 160 is also comprised of at least one temperature sensor 164 that measures the pedal temperature of the patient through one or each of the patient's feet 12. The pedal temperature is preferably measured in Fahrenheit or Celsius. The pedal temperature detected is communicated to the display 110 via the wire 150 in the form of an electrical signal. The electrical signal is then converted by at least one processor 170 of the display 110 into a temperature that is displayed 126,136 on either or both foot display area 120,130.

In one embodiment, the pedal pulse oximetry sensor 162 and pedal temperature sensor 164 communicate wirelessly with the processor 170. In this embodiment, both sensors 162,164 are comprised of at least one transmitter 194. Each transmitter 194 is in wireless electrical communication with at least one receiver 196 of the processor 170 via Wi-Fi, Bluetooth, RFID, etc. In this manner, no wires 150 of the device 100 can become tangled during use. It should also be appreciated that the device 100 can monitor the pedal pulse rate, pedal pulse quality and pedal temperature of each foot 12 of the patient unilaterally or bilaterally.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “pedal pulse monitoring device” and “device” are interchangeable and refer to the pedal pulse monitoring device 100 of the present invention.

Notwithstanding the foregoing, the pedal pulse monitoring device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the pedal pulse monitoring device 100 as shown in the FIGS. are for illustrative purposes only, and that many other sizes and shapes of the pedal pulse monitoring device 100 are well within the scope of the present disclosure. Although the dimensions of the pedal pulse monitoring device 100 are important design parameters for user convenience, the pedal pulse monitoring device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A pedal pulse monitoring device comprising:
a display;
a pad;
a pulse sensor;
a temperature sensor; and
a wire connected to the display and the pad, wherein the wire places the pad in electrical communication with the display.

2. The pedal pulse monitoring device of claim 1, wherein the display is comprised of a touch screen, an LCD screen, an OLED screen, an ELD screen, an LED-backlit LCD screen, an LED screen or a PDP screen.

3. The pedal pulse monitoring device of claim 1, wherein the pulse sensor communicates a pulse to the display, and further wherein the temperature sensor communicates a temperature to the display.

4. A pedal pulse monitoring device comprising:
a display comprised of a processor, a battery and a fastener;
a pad comprised of a temperature sensor and a pulse sensor having an electrode; and
a wire connected to the pad and to the display, wherein the wire places the pad in electrical communication with the display.

5. The pedal pulse monitoring device of claim 4, wherein the pulse sensor detects a pulse via the electrode, and further wherein the electrode produces an electrical signal from the pulse.

6. The pedal pulse monitoring device of claim 5, wherein the electrical signal is relayed to the processor where it is converted into a beats per minute unit that is then displayed on the display.

7. The pedal pulse monitoring device of claim 4 further comprising a speaker.

8. The pedal pulse monitoring device of claim 4 further comprising a light.

9. A pedal pulse monitoring device comprising:
a display;
a processor;
a battery;
a receiver;
a fastener;
a speaker;
a light;
a pad;
a pulse sensor positioned on the pad and comprising an electrode;
a first transmitter; and
a temperature sensor comprising a second transmitter.

10. The pedal pulse monitoring device of claim 9, wherein a bottom surface of the pad is comprised of an adhesive bottom surface.

11. The pedal pulse monitoring device of claim 10, wherein the adhesive bottom surface is comprised of a skin safe adhesive.

12. The pedal pulse monitoring device of claim 9, wherein the fastener is comprised of a clip, a clamp, hook and loop, a magnet, a suction cup, a threaded bolt, a hinge, or a swiveling fastener.

13. The pedal pulse monitoring device of claim 9, wherein the display is comprised of a charging port.

14. The pedal pulse monitoring device of claim 13, wherein the charging port receives a reciprocating charging cord of a charger.

15. The pedal pulse monitoring device of claim 9, wherein the first transmitter and the second transmitter are in wireless electrical communication with the processor.

16. The pedal pulse monitoring device of claim 9, wherein the display is comprised of a first foot display area that displays a first pulse with a first pulse category from the pulse sensor and a first temperature from the temperature sensor.

17. The pedal pulse monitoring device of claim 9, wherein the temperature sensor communicates a temperature signal to the display in the form of an electrical signal.

18. The pedal pulse monitoring device of claim 17, wherein the processor converts the electrical signal into a Fahrenheit or Celsius unit.

19. The pedal pulse monitoring device of claim 16, wherein the speaker and light are activated when the first pulse category is detected as an absent pulse, a weakened pulse, or a bounding pulse.

20. The pedal pulse monitoring device of claim 19, wherein the light rapidly flashes.
